(19) United States
(12) Patent Application Publication
(10) Pub. No.: US 2013/0343953 A1
(43) Pub. Date: Dec. 26, 2013

(54) TREATMENT OF FLUID-BORNE CANCER CELLS

(71) Applicant: Eric V. Kline, Rochester, MN (US)

(72) Inventor: Eric V. Kline, Rochester, MN (US)

(73) Assignee: INTERNATIONAL BUSINESS MACHINES CORPORATION, Armonk, NY (US)

(21) Appl. No.: 13/532,329

(22) Filed: Jun. 25, 2012

(51) Int. Cl. A61M 1/36; B01D 29/52; B01D 29/90; B01D 29/60; B01D 15/38; B01D 37/00; A61M 1/16; B03C 1/30

(52) U.S. Cl. 422/48; 422/44; 210/435; 210/416.1; 210/323.1; 210/223; 210/138; 210/407; 210/180; 210/198.2; 210/767; 210/806; 210/695; 210/808; 210/774; 210/748.02; 210/748.09; 210/748.01; 210/656

(57) ABSTRACT

Provided are devices, device systems, and methods to intercept and kill fluid borne cancer cells to slow or prevent metastases.

### BACKGROUND OF THE DISCLOSURE

Cancer cell metastatic progression may be substantially supported by solid-fluid interaction, such as movement of cancer cells in blood (hematogenous spread), lymph (lymphatic spread), or cerebro-spinal fluid. While cancer cells are suspended in such biological fluids they are potentially vulnerable to clinical therapies, thereby reducing or even arresting metastasis. It is noted by many, though not universally medically accepted, that surgery to remove a tumor can be a significant source of metastasis, since cancer cells in the tumor are disturbed during the process and release of tumor cells during bleeding is likely to occur.

### BRIEF SUMMARY OF THE DISCLOSURE

Provided are devices, device systems, and methods to intercept and kill fluid borne cancer cells to slow or prevent metastases.

In one example, this disclosure provides a filtration device for filtering fluid-borne cancer cells from the biological fluid of a subject with cancer. The device includes a filter; an input valve for delivery of biological fluid from the subject into the filter; a first filter output valve from the filter to return the filtered biological fluid back to the misfortunate subject; and a second filter output valve from the filter for channeling waste cells and materials trapped by the filter away from the fluid to be returned to the subject.

The device can also include at least one pump for moving the biological fluid through the filter. The fluid can be blood. The mesh size of the filter can be between 10 to 30 microns.

The device can include more than one filter. The device can contain at least two parallel filtration chambers with time-alternating filtration and waste destruction functions such that at least one filter is operational at all times.

In another example, the device can further include a deoxygenation chamber to deoxygenate cells prior to filtration. For example, the input valve of the device can connect first to a deoxygenation chamber that then connects to a magnetic field chamber. Biological fluid passing into the deoxygenation chamber is deprived of oxygen, then the fluid passes into the magnetic field chamber where it is subjected to magnetic force sufficient to separate paramagnetic cells, such as red blood cells, from non-paramagnetic cells and other materials. The magnetic field chamber can have a first output valve connected to the filter to deliver biological fluids to the filter, in addition to a second output valve for channeling non-paramagnetic cells and materials away from the biological fluid. In this manner, biological fluid is subjected to deoxygenation followed by magnetic separation of non-paramagnetic cells and materials from said biological fluid prior to delivery of said biological fluid into the filter or filters.

There can further be a cooling system integrated between the deoxygenation chamber and the magnetic field chamber, which system cools the deoxygenated biological fluid prior to passage into the magnetic field chamber. Alternatively, or in addition, an oxygenation chamber is connected to the first output valve of the magnetic field chamber to re-oxygenate said biological fluid following magnetic separation.

This disclosure further provides for homogenization of waste cells and materials trapped by the filter or filters, the homogenization being achieved by administration of one or more of ultrasonic pulses, heat, cold, radiation, electrocution, and mechanical milling or grinding to the waste cells and materials trapped by said filter.

The filtration device according to this disclosure can further include a chamber for damaging mitotic cells before or after filtration of the biological fluid. For example, the valve delivering biological fluid from the subject can connect first to a mitotic cell damaging chamber having an output valve connected to the filter, such that the biological fluid is subjected to mitotic cell damaging treatment prior to delivery of said biological fluid into said at least one filter. Alternatively, the mitotic cell damaging chamber can be attached to the filter output valve, such that the biological fluid is subjected to mitotic cell damaging treatment after filtration of the fluid. The mitotic cell damaging treatment can be selected from one or more of radiation, UV light, radiowave frequencies, ultrasonic pulses, heat, cold, or hypoxia.

This disclosure further provides a device for filtering fluid-borne cancer cells from the biological fluid of a subject with cancer which includes an affinity column that binds one or more cancer antigens; an input valve for delivery of biological fluid from the subject into the column; and an output valve from the affinity column to deliver the filtered biological fluid back into the subject. This device can include at least two such affinity columns operating in parallel. Further, the output valves of the at least two parallel affinity columns can connect to at least two parallel filtration chambers with time-alternating filtration and waste destruction functions, such that the biological fluid is subjected to affinity purification followed by filtration, where at least one filter is operational at all times.

This disclosure further provides microscale versions of any of the disclosed devices, integrated on silicon or other semiconducting substrate.

This disclosure also provides methods for filtering fluid-borne cancer cells from the biological fluid of a subject with cancer, the methods comprising filtering the biological fluid of said subject through a filtration device and returning filtered biological fluid back to said subject, wherein the device may be any of the devices described herein.

### BRIEF DESCRIPTION OF THE FIGURES

FIG. 1. Example of Type C Cancer Cell Killer.

FIG. 2. Single Stage Passive Type A Filtration Device. A passive single stage filter with a filtration size of about 20 microns separates out plasma and RBCs from larger cells which collect in front of the filter. A waste value is illustrated to permit removal of waste cells (which could include cancer cells and well as larger non cancerous cells which normally are seen in blood or other biologic fluids) for sampling or discard purposes.

FIG. 3. Multi-Stage Passive Type A Filtration Device.

FIG. 4. Type B-1 Active Filtration Device.

FIG. 5. Type B-2 Active Filtration Device.

FIG. 6. Type C Device.

FIG. 7. Type D (Selective Cancer Cell Binding) Device.

FIG. 8. Type E Hybrid Device. The preferred example includes a combination of multiple invention types, such as Type A or B-2 with Type C or D. Type E examples represent a two stage separation-killing function, providing added assurance no cancer cells would be reintroduced back into the patient.

FIG. 9. Type F Doctor on a Chip Device.

FIG. 10. Type F Doctor on a Chip Device.

### DETAILED DESCRIPTION OF THE DISCLOSURE

Since much metastatic disease progression in cancer is associated with biological fluid transport of cancer cells, circulating metastatic cells are highly accessible for therapeutic intervention designed to slow or halt the progression of metastasis. In contrast, red blood cells (RBCs) and other non-cancerous blood borne cell types undergo mitosis less frequently and are therefore more impervious to mitosis disrupting treatments such as light, ultrasonics, and chemotherapeutic agents. This disclosure is based on the finding that cells carried by a biological fluid can receive high doses of anti-cancer treatment by subjecting the biological fluid to anti-cancer treatments while the fluid is removed from the subject. The treated fluid is then re-introduced to the subject, thus treating fluid-borne cancer cells without significantly compromising internal body tissues. Blood cell cancers, such as leukemia and various lymphomas, are particularly vulnerable to treatments as disclosed herein. The terms “treated”, “filtered”, and “processed” are used interchangeably herein.

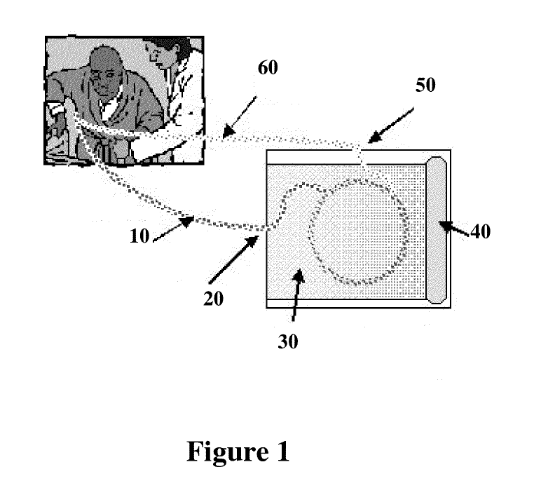

This disclosure presents devices and device systems, and methods to intercept fluid borne cancer cells and clinically treat them to damage or kill the cancer before it has an opportunity to spread. An example of the disclosed devices is illustrated in FIG. 1 wherein the device is shown as an in-series shunt of a patient's blood flow in an artery or vein. Blood contaminated with cancer cells or “contaminated blood” 10 is drawn from the patient. The contaminated blood flows through an input valve 20 and is subjected to anticancer treatment 30 such as a transducer 40 that subjects the blood to light, ultrasonic waves, or other treatments. The “decontaminated blood” 60 which contains no or reduced numbers of viable cells exits the treatment device through an output valve 50 for return to the patient. Devices such as shown in FIG. 1 can be administered internally or as a permanent or temporary peripheral appliance. The devices can be practiced in several different modes, such as a mobile device which is worn by the patient, or a bedside device to which the patient is attached periodically.

The devices disclosed herein separate cancer cells from RBCs and non-cancer cells. This separation can be based, for example, on differences in cell size, volume, mass, membrane properties, magnetic pull, antigenic determinants, protein or receptor expression, and combinations thereof. The devices can provide, or be can be operated in conjunction with other devices that provide, cancer cell damaging agents including but not limited to radiation; infrared, ultraviolet or visible light; x-rays; ultrasonic or radiofrequency waves; hot or cold temperatures; radionuclides; high pressure; anaerobic conditions; mechanical homogenization; and combinations thereof.

Specific examples of devices according to the disclosure are as follows. Methods of use of the disclosed devices are evident to those of skill in the medical arts. The uses generally involve accessing biological fluid of a subject, such as through a vein or artery, circulating the fluid through the device in a sterile manner, and returning the filtered biological fluid to the subject, such as into a distant or adjacent vein or artery.

Type A Devices.

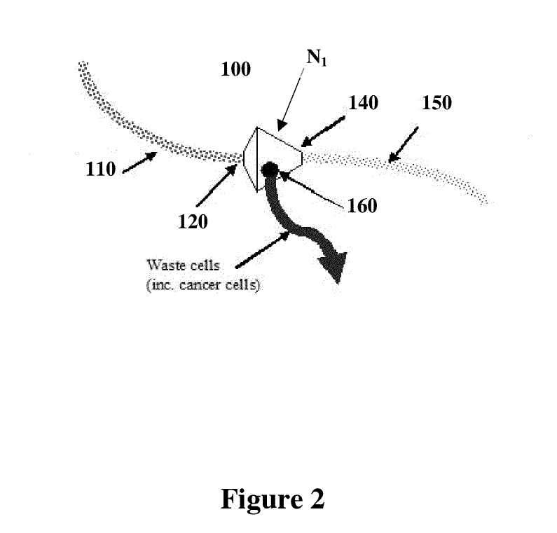

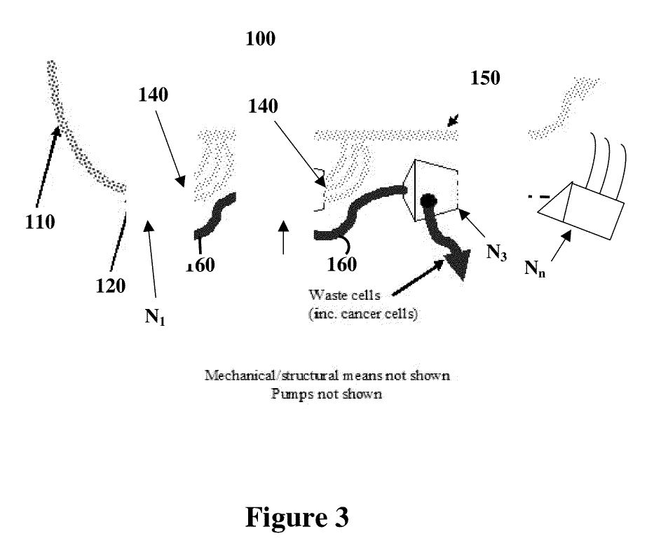

Type A devices, as in FIGS. 2 and 3, employ passive filtration to separate cancer cells from non-cancerous cells based on cell size. Type A devices employ replaceable, sterile filters which allow RBCs (and other blood or fluid borne small cells) to pass while trapping large cells (including eosinophils, macrophages, T and B cells, and cancer cells). The device 100 has an input valve 120 for uptake of contaminated blood or lymph 110 from the subject, which delivers the biological fluid through an input valve 120 into a filter mechanism N₁ for filtration. The fluids are filtered through the filtration mechanism and filtered, decontaminated fluid 150 is returned to the subject through an output valve 140. Fluids can be filtered through a single filter N₁ (single stage filtration) or multiple filters N₁, N2, N3 . . . N. (multi-stage filtration) prior to re-introduction into the subject. The filter or filters can have a mesh/pore size of 0.1, 1, 5, 10, 15, 20, 25, 30, 40, 50, 60, 70, 80, 90, or 100 or more microns. The device can include one or more pumps to move cells and fluid from the subject through the filter or filters and to return fluids back to the subject. Trapped large cells pass into a waste valve 160 for sampling or discarding, or alternatively are cycled through the multi-stage (serial or mixed serial and parallel) version to increase recycling of RBCs and plasma back to the patient. Examples of Type A filtration modes are shown in FIG. 2 (single stage filtration) and FIG. 3 (multi-stage filtration).

The multi-stage passive Type A filter system operates in the same way as the single stage passive filter with the exception that waste cells from early filter stages are input to the next filtration stage, where additional RBCs are collected for return to the patient. No limit is implied to the number of passive sequential filtration stages. In addition, multiple RBC separation or collection ports can be provided in each filtration stage.

Type B Devices.

Type B devices encompass the passive filtration of Type A devices, plus active filtration, with two Type B sub-types provided.

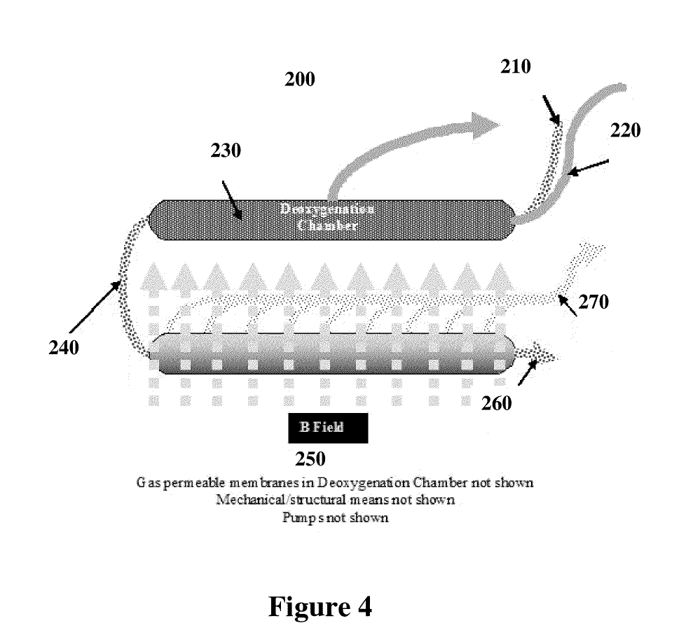

A Type B-1 device 200 is exemplified in FIG. 4. In one example, contaminated biological fluid 210 containing red blood cells (RBCs) is deoxygenated in a deoxygenation chamber 230 which then moves through a second input valve 240 and is then subjected to a magnetic field in a magnetic chamber 250 prior to filtration. In one example, RBCs are deoxygenated by employing mild pressure on the vessel containing the biological sample, and/or by purging of the sample vessel with N₂ or other inert gas (such as Argon or Helium) through a gas input valve 220, which would gradually displace the oxygen from the hemoglobin, rendering deoxyhemoglobin-containing RBCs. The deoxyhemoglobin has a paramagnetic moment (μ=5.46 uB) which can be used to separate out RBCs from other non-hemoglobin containing cells by application of an external magnetic field, as illustrated in FIG. 4, prior to passing through a passive filtration means/Type A-type filtration device (not shown). Processed blood 270 passing through the Type B-1 device would contain essentially RBCs (plus other small cells) in plasma for return back to the patient while large cells are discarded as waste through a waste valve 260.

An integrated optional cooling system is further provided to cool the deoxygenated blood prior to magnetic field separation 1° C. to 20° C., preferably between 1° C. to 10° C.], to enhance the relative paramagnetic susceptibility of the deoxyhemoglobin for more efficient separation of RBCs from non-RBCs. Another optional aspect is a chamber or system providing reoxygenation to deoxygenated RBCs prior to reintroduction back into patient. Such an oxygenation system can be placed between the magnetic field chamber and the filter, after the filter, or integrated into the filter.

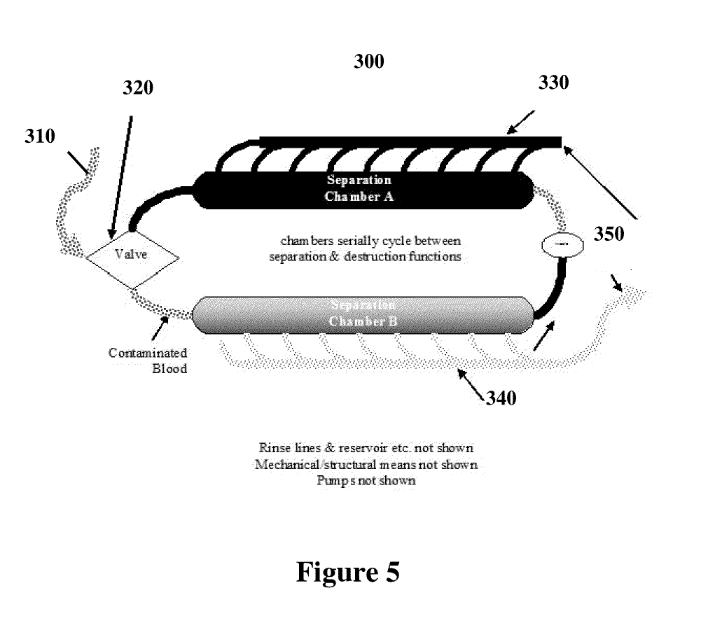

The Type B-2 device, as illustrated in FIG. 5, provides passive filtration with increased waste cell homogenization. Homogenization of waste cells aids in prevention of the passive filter means from clogging and also enables operation without time limit. Type B-2 devices employ parallel filtration chambers with time-alternating filtration-waste destruction functions to ensure at least one chamber is operating in filtration mode at all times. This unit can be self-contained, returning destroyed cell matter back to the patient's blood stream alongside viable RBCs and plasma, or alternatively the unit can be configured to dump waste cell debris into a waste stream which does not return to the patient. The various means to achieve waste cell destruction include strong ultrasonic pulses (such as pulses at low power), heat (40° C. or above, preferably between 43° C. and 50° C.), cold (0° C. and below, preferably between −10° C. and −100° C.), radiation (such as 0.001 to 10 Gy per minute, preferably between 0.1 to 2 Gy per minute), electrocution (such as electric pulses between 10 mA to 1000 mA), and mechanical milling or homogenization. FIG. 5 illustrates a Type B-2 device 300. Contaminated blood 310 passes through an input valve 320 that alternately cycles fluids into separation chamber A 330 or separation chamber B 340. Decontaminated blood passes through output valves 350 for return to the patient. It is observed in FIG. 5 that parallel treatment chambers are employed which cycle in time, alternating between their two functions: (1) separation of RBCs, and (2) destruction of large cells entrapped by filter. The purpose of the dual function is so the chamber can rid itself of large cellular debris which builds up after some prolonged period of operation, so that it can maintain efficiency. The purpose of having two parallel chambers, which can function alternatively, is to ensure that the RBC separation function can proceed unhindered, returning needed RBCs and plasma back to the patient at all times. It will be observed that rinse lines, pumps, reservoirs, and other optional features of the device are not shown in FIG. 5.

The general method of Type B-2 operation is: (1) separation of RBCs and monitoring of delta filter pressure; (2) regular system-initiated checks of each chamber status to ensure at least one chamber is in and will remain in separation mode for some minimum fixed duration; (3) for any chamber requiring removal of built up debris, configuration of destruction mode (patient blood or biologic fluid feed is turned off, cell destruction means and waste streams are turned on at appropriate times); (4) the chamber requiring clearance executes cell destruction features (turning on ultrasonic, heat, electrodes, radiation source, or other homogenization source); (5) once waste cells are homogenized, the waste stream is opened for return to patient, sampling for pathology, or to biological waste; and (6) the system initiates reconfiguration of the cleared chamber for further RBC separation and recovery function.

Type C Devices.

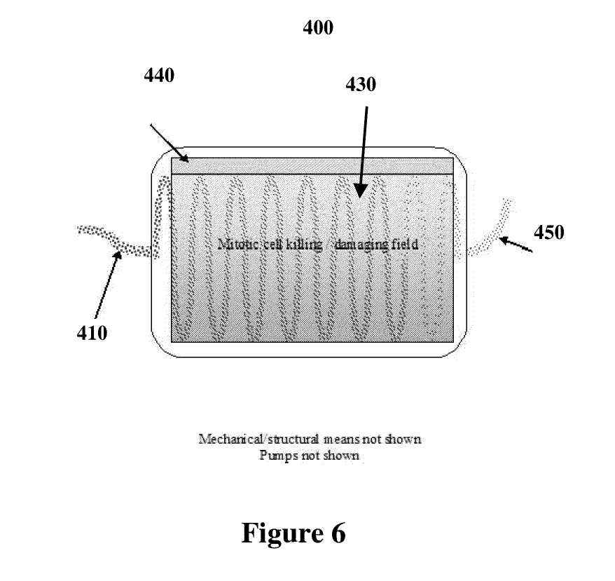

Type C devices apply general mitotic cell killing treatments to all cells in biological fluid sample. Mitotic cells are vulnerable to a variety of biologic stressors such as ultraviolet (UV) light (such as wavelength of 260 nm to 280 nm, or irradiance at 1 to 20 Joules/square meter), ultrasonic pulses (such as pulses at 10 W power), heat (40° C. or above, preferably between 43° C. and 50° C.), cold (0° C. and below, preferably between −10° C. and −100° C.), radiation (such as 0.001 to 10 Gy per minute, preferably between 0.1 to 2 Gy per minute), electrocution (such as electric pulses between 10 mA to 1000 mA), and/or hypoxia. Thus, as seen in FIG. 6, the Type C device 400 uses mitotic cell vulnerability to apply a cancer cell damaging regime to a contaminated blood or biological fluid sample that enters the system 430 through an input valve 410. Inside the device, a transducer 440 applies a biologic stress to the fluid that selectively kills dividing cells while maintaining manageable levels of collateral cell damage. All cells so treated are available for return back to the patient through an output valve 450 or can be sent to waste collection. These devices employ continuous fluid sampling combined with one or more mitotic cell killing techniques to selectively kill dividing cells. RBCs and many other naturally occurring blood borne cells do not divide, and are thus substantially immune to such treatments, which include radiation (especially Beta radiation, using such isotopes as ¹³¹I), UV, Visible, IR light, RF, ultrasonic pulses, heat, cold, hypoxia, and other means cited in Table 1. Filtration of cells by any of the above filtration methods described above, prior to or subsequent to the DNA damaging treatment, is a further aspect of this example.

Cells subjected to DNA damage try to repair such damage immediately after cellular insult. The first few hours after damage is the most critical time-frame for repair. Hypoxic conditions can reduce or prevent cell repair. Thus, a combination of cell killing treatments includes a first exposure to a killing regime (i.e., ultrasonics, electrocution, radiation, heat, etc.) followed by immediate exposure to hypoxic conditions to ensure no cancer cell survival. In addition, hypoxia by itself can kill rapidly dividing cancer cells as mitotic cells require cellular respiration at a greater level than non-mitotic cells. Thus, exposure of a biologic fluid stream in a vessel to hypoxic conditions, for a period of at least one hour, can damage mitotic/cancer cells while allowing return of the treated biological fluid back to the patient. As an optional step, reoxygenation of patient fluid samples following hypoxic treatment corrects deoxygenated RBCs prior to reintroduction back into the patient.

Type D Devices.

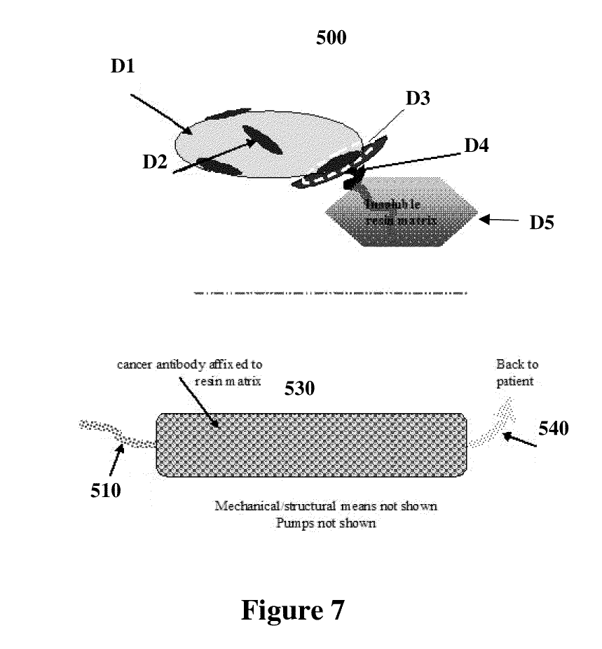

Type D devices, as shown in FIG. 7, remove cancer cells from subject fluids by exposing the fluid to cancer-cell-specific binding molecules, separating the fluid from the molecules, and removing and destroying cancer cells. A preferred method for separating cancer from non-cancerous cells in blood or biologic fluids is via an immobilized antibody specific to the cancer cell type. Cancer cell-specific antibodies and methods of generating such antibodies are known in the art. In a preferred example, cancer cell-specific antibodies are generated employing Enhanced Antigen Expression methods as disclosed in U.S. patent application Ser. No. 13/302,552, the contents of which are incorporated by reference in its entirety.

In a Type D system 500 as shown in FIG. 7, the anti-cancer antibody D4 would be chemically bonded to a non-soluble (in aqueous fluid) porous resin matrix affinity column D5, such that the active antibody portion would be exposed and available to attachment to a cancer cell-specific antigen D2, D3 on the surface of the cancer cell D1. Numerous cancer cell antigens and cancer antigen-specific antibodies are known in the art, for example, antibodies to the cancer antigens CA125, TMEFF2, SPAS-1, etc. Sterility and biocompatibility of the column, its components, valves, etc would be maintained to ensure the biological fluid returned to the patient is free from contamination and toxicity. The blood or other biologic fluid 510 would be pumped through the column 530 by an input valve, cancer cells would be captured in the column, and non-binding cells and plasma would flow through the column out an output valve 540 and be returned to the subject. The cancer cells would then be eluted from the column and destroyed. The column affixed with anti-cancer antibodies would then be reconditioned and reused. FIG. 7 illustrates the Type D example and method. The Type D example can be configured with two (or more) columns in parallel so that cancer cell binding and subsequent rinse operations can be alternatively off-set in time.

The general method of Type D operation would be (single column example): (1) filter cancer cells from biological fluid using a column comprising cancer-cell-specific antibodies, wherein the cancer cell would be reversibly bound to the column; (2) the filtrate is returned to the subject; (3) the fluid stream to the column is periodically shut off and the cancer cells eluted from the column using pH, salinity, EDTA or other known techniques; (4) the rinse filtrate is discarded and/or sampled for pathology; and (5) the antibody impregnated resin column would then be reconditioned with proper pH, salinity, etc. to render the column ready to resume normal cancer cell binding operation.

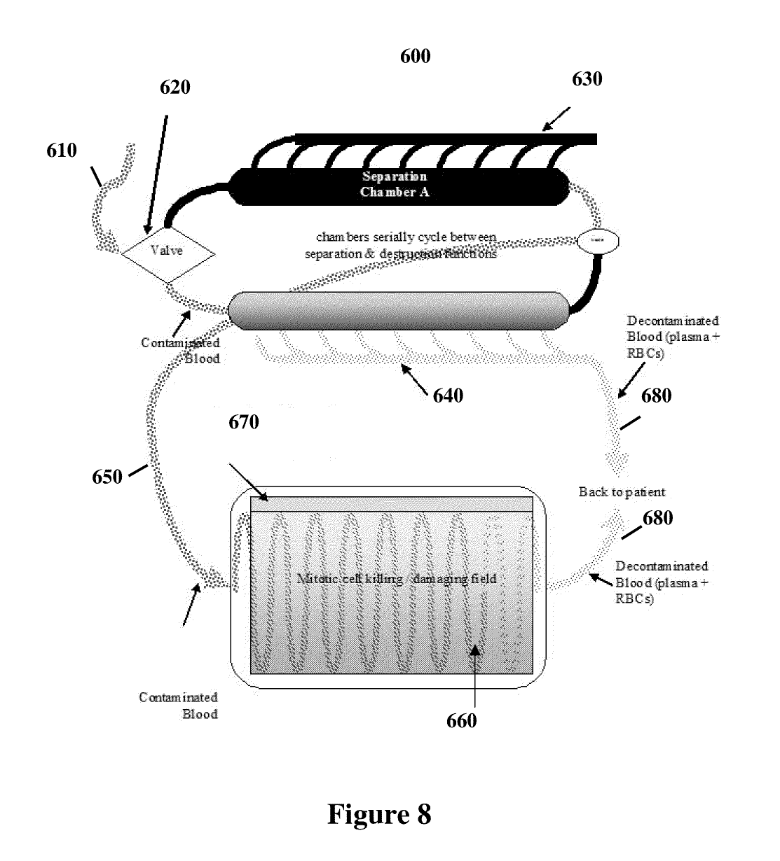

Type E Devices. Type E devices combine aspects of Type B devices with either Type C or D devices. FIG. 8 illustrates a Type E Hybrid device 600. In this example, contaminated blood 610 passes through an input valve 620 that alternately cycles fluids into separation chamber A 630 or separation chamber B 640. After passing through the first stage separation, the blood travels to a second input valve 650 leading to a chamber for a second treatment 660. The second treatment chamber 660 applies a second treatment to damage mitotic cells in the blood by means such as a transducer element 670. Decontaminated blood passes through output valves 680 for return to the patient. As in FIG. 5, the parallel treatment chambers 630, 640 cycle in time, alternating between their two functions: (1) separation of RBCs, and (2) destruction of large cells entrapped by filter. Thus, Type E devices provide a two stage separation-killing function.

Type F Devices.

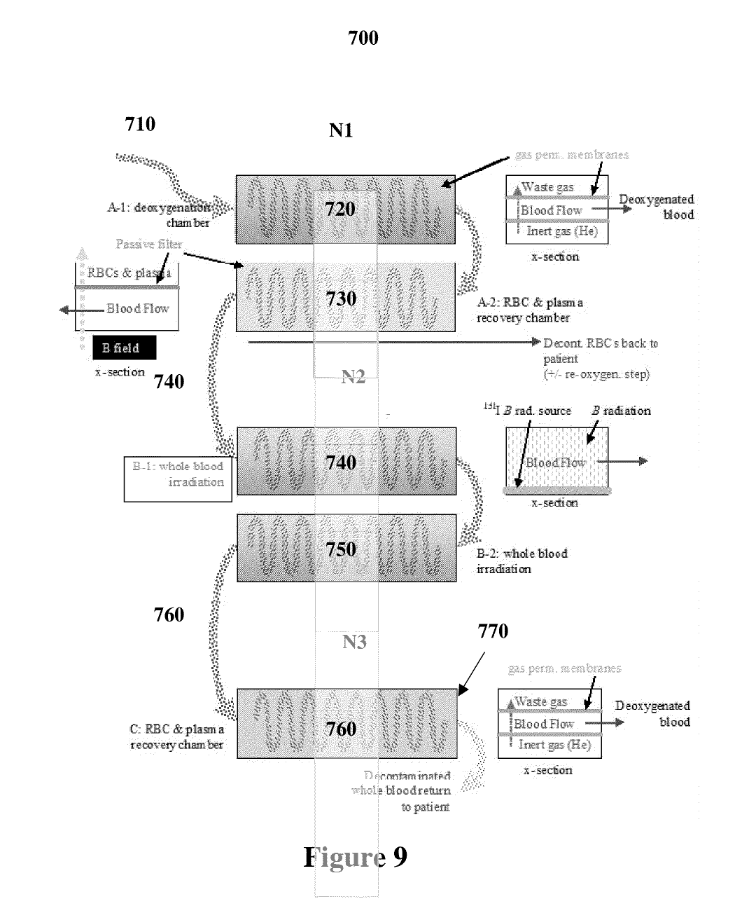

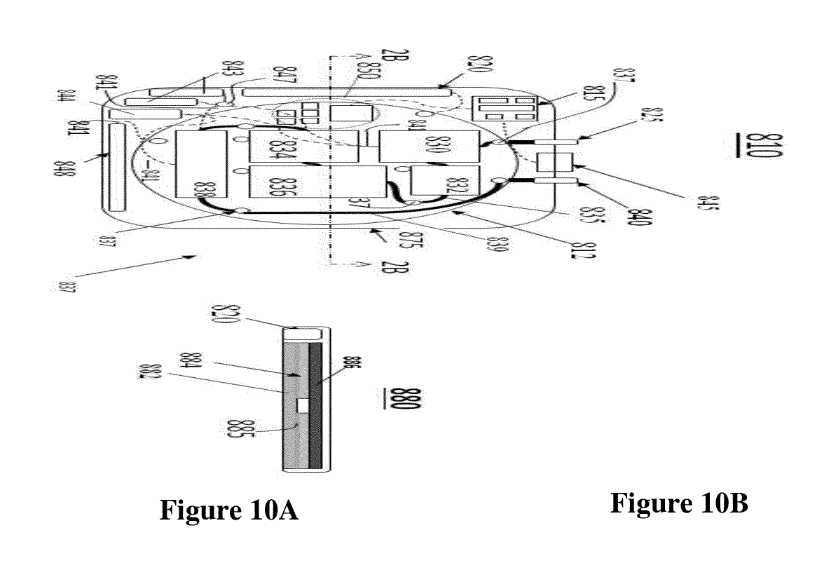

Type F devices integrate any of Type A thru E integrated on silicon or other semiconducting substrate as a “Doctor on a Chip” (DoC) for leverage of electronic, electrical or MEMS capabilities in a miniaturized system. The advantages of this example include self-contained operation and ability to embed monitoring, control and analytics functions within the system. FIG. 9 illustrates a functional block diagram for a Type F example 700. In this example, multiple blocks N₁, N₂, N₃ . . . Nₙ apply multiple treatment steps within a self-contained micro-apparatus. Although FIG. 9 provides one example, it is understood that any combination of Type A, B-1, B-2, C, D, and/or E devices can be incorporated, in any order, in the DoC. In FIG. 9, contaminated biological fluid 710 containing red blood cells (RBCs) moves through Block A N₁, which provides for RBC recovery via magnetic susceptibility as in a Type B1 device. In Block A N₁, fluid is deoxygenated in a deoxygenation chamber 720 and is then subjected to a magnetic field in a magnetic chamber 730 prior to moving to Block B N₂, which provides for RBC recovery via filtration and irradiation of large cells. In Block B N₂, one or more transducers 740, 750 apply radiation, such as ¹³¹I radiation, to selectively kill dividing cells in the fluid, as in a Type C device. Filtration of cells by a microfilter, prior to or subsequent to the DNA damaging treatment, can also be incorporated into the device. Following Block B treatment, the processed fluid is subjected to hypoxic treatment in Block C N₃. In Block C, a deoxygenation chamber 760 applies hypoxic conditions, as in the deoxygenation chamber 720 of Block A. Here, hypoxic treatment of cells limits or prevents DNA repair, thus further damaging mitotic cells. Once the fluid has processed by moving through the DoC, the fluid is returned to the patient by an output valve 770. Self-contained packaging of a Type F device can create a DoC in a Box (DiB) as seen in FIGS. 10A-10B.

FIG. 10A shows an embodiment of an implantable DiB module 810 (“module”). In particular, module 810 includes a bio-compatible package 875 housing components such as: a semiconductor material substrate (e.g., silicon or type III-IV material substrates) wafer or substrate 812 having formed thereon a plurality of systems including elements (e.g., active and passive device) configured to perform the sensing and active driving functions; and, a power supply 815 including a power source such as a battery 820 for providing the power to the configured elements. Further, included in module 810 is an external connector 845 adapted to provide power (e.g. electric current) to the power supply device within packaging 875 from an external power source, e.g., battery, generator, etc. for performing DiB functions. The external connector 45 is an electrical connector for wire based communication between module and external node. The connector 45 can also be a mechanical connector for fluid communication, for example, to accept fresh reagents and/or flush wastes. The DiB module 810 may be of a size on the order of less than about 30-50 mm on a side housing the wafer 812 and additional configured elements. The DiB module may contain additional elements as described in copending U.S. application No. ______, filed ______, the contents of which are herein incorporated by reference.

The packaging 785 can have a coating of a bio-compatible metal, such as alumina, zirconia, or alloys of alumina and/or zirconia, or other bio-compatible substances such as poly(ethylene glycol) or polytetrafluoroethylene-like materials with thicknesses ranging from between 0.5 μm and 20 μm. The biochips are on the order of 10 to 50 mm on a side, with the coating/membrane thickness between 20 and 800 microinches. Alternatively, the packaging can be a composite material including but not limited to insulating materials, water-vapor permeable materials, and polymeric materials, such as epoxies, urethanes, silicones, resins, Parylene, and the like. The packaging can be a polymer comprising hydrophobic, hydrophilic, or amphipathic molecules, proteins, peptides, cell membrane components, and/or other biological components on the packaging exterior (see for example U.S. Pat. Nos. 5,589,563, 6,770,725, 7,884,171, 8,012,587, and 8,005,526, the entire contents of which are herein incorporated by reference). The packaging can further contain anti-microbial compositions and/or be sterilized (see for example U.S. Patent Publication Nos. 20050008676, 20100285084, 2009023270, the entire contents of which are herein incorporated by reference).

In one example, the configured elements on a surface of a substrate or wafer 812 include MEMS (Micro-Electro-Mechanical Systems) devices, and other semiconductor devices, such as active and passive semiconductor devices and systems. The MEMS cantilever arm is situated on the external surface of the outer layer so that the arm can extend out externally from the package.

Further provided is an input connector device 825 adapted for receiving a patient's biological fluid. Such input connectors are known in the art. The input connector 825 permits biological fluid or fluid communication with a patient's tissue. An optional membrane or screen filter can be included to keep the device from clogging, in which case the pump could, periodically or under pressure monitoring, reverse flow to relieve the membrane/screen of debris. Input connector 825 is in fluid communication between patient's biological fluid and/or tissue and a micro-fluidic pump element 837 mounted within or on wafer 823 within packaging 875 for receiving the biological fluid and pumping the biological fluid via channels to a series connection of interconnected chambers or reaction wells 830, 832, 834, 836, 838 within which chemical reactions, filtering, analysis and/or other treatment of the biological fluid is performed.

At least one barrier layer covers each chamber, to isolate the reacting component from biological components outside the chambers. The barrier layer can be selectively disintegrated or permeabilized to expose the reacting component to the biological fluid.

For example, to perform a blood processing step, the barrier layer on the chamber is removed or made permeable under control of the CPU. The biological fluid passes into the chamber 830 (by diffusion or active pumping by pump element 837 of fluid into chamber), the processing/filtration step or steps are performed, and the processed fluid moves out of the chamber 830 to a fluid output 840 connector. This process may be repeated numerous times by opening additional chambers 832, 834, 836, 838 for fluid processing.

Inter-connecting each fluid chamber 832, 834, 836, 838 is a conduit structure, such as fluid micro-channels 835, and one or more micro-pumps 837. Inter-connections can be parallel connections, serial connections, or combined serial and parallel connections. An output fluid micro-pump 837 and output fluid channel 839 provides output fluid material from chamber 838 to a fluid output 840 connector, which outputs biological fluid, waste, or other materials, either into the patient tissue or adjacent fluid system (blood or lymphatic system), or connected to other biological fluid output for further analysis within or external to the patient.

Further included within packaging 875 of module 810 is one or more containers 843, e.g., a vessel or well, for carrying a gas, a chemical, or a reagent, each of which are connected to reaction chambers 832, 834, 836, 838 via microchannels 841 in fluid connection with a pump 847 for pumping controlled amounts of reactants/reagents necessary to perform a processing step from, e.g., a reactant vessel or tank 848. A control device, a central processing unit (CPU) or controller controls the actuation of the micro-pumps 837 such that a reagent, e.g., a gas or chemical from a well 843 or like vessel, may be released or transported into a reaction well or chamber. A further waste storage vessel or well 844 is in further fluid communication with chambers 832, 834, 836, 838 via microchannels 841 for receiving waste products or other materials, e.g., fluids, gases, from reactions. The CPU or controller may control the configuration of connections of fluid channels to/from the micro-pumps to reaction chambers 832, 834, 836, 838 and control the timing of reactive and reaction product material transfers to/from reaction chambers 832, 834, 836, 838. Chamber sizes can range from 1 cc to 300 cc, preferably between 10 cc to 100 cc.

In one embodiment, the CPU may be used to initiate a controlled release of a material, e.g., medicine, therapeutic agent, etc.

In one embodiment, configured elements formed on substrate 812 include an Integrated Circuit (IC) controller unit or system 850 for controlling on-chip functions/operations. Such IC controller unit 850 includes on-chip components formed on a wafer substrate including but not limited to: a processing core including a Central Processing Unit (CPU) or like programmable controller and associated cache memory, a memory system including a Random Access Memory (RAM). The memory system alternately, or in addition, may include DRAM or SRAM devices, or both, or other memory devices such as flash and EEPROM.

FIG. 10B shows a cross-sectional view of DiB module 810 taken from line 2B-2B of the module 810 shown in FIG. 10A. In one embodiment, DiB module 810 is a stacked arrangement 880 including a first wafer layer 882 providing the interconnecting electrical conductor layers for routing of power, I/O signals control signals, etc. and power signal routing network for delivering power to configured elements of substrate 812. DiB module 810 additionally includes a second wafer layer 884 having the formed reaction chambers 830, 832, 834, 836, 838, the reactant stores and waste product vessels, fluidic interconnects 835 and micro-pumps 837. DiB module 810 further includes a third wafer layer 886 having configured elements such as the CPU, memory, and MEMs circuits and devices. The wafer may further include a container of radioactive materials, for example a radionuclide.

Type F device functional blocks, described in connection with FIG. 9, are listed in Table 1. “RBC recovery” includes plasma recovery. It is noted that an optional re-oxygenation of RBCs step can be included by adding another functional block which reverses the Block A (A-1) process step, for example, replacing Helium with ‘air’ or oxygen.

TABLE 1
Type F Functional Blocks for FIG. 9

| | | Device | |
|---|---|---|---|
| BLOCK | Function | Type | Resources |
| A | RBC recovery | B-1 | deoxygenation chamber (A-1), gas |
| | via magnetic | | permeable membrane gas supply |
| | susceptibility | | (Ar, He, N2, etc.), RBC |
| | | | separation & recovery (A-2), |
| | | | magnetic field, RBC passive filter |
| | | | means, fluid recovery stream, |
| | | | optional re-oxygenation step |
| B | RBC recovery | B-2 | one or more irradiation chambers, |
| | via filtration + | | control valves (input and output), |
| | radiation of | | filter means, fluid RBC recovery |
| | large cells | | stream |
| C | Whole blood | C | modified A-1 type deoxygenation |
| | recovery via | | chamber, gas permeable inert |
| | hypoxia | | gas supply, fluid recovery, |
| | treatment | | optional re-oxygenation step |

What is claimed is:

1. A filtration device for filtering fluid-borne cancer cells from the biological fluid of a subject with cancer, the filtration device comprising: (a) at least one filter; (b) an input valve for delivery of biological fluid from said subject into said at least one filter; (c) a first filter output valve from said at least one filter to return the filtered biological fluid back into said subject; and (d) a second filter output valve from said at least one filter for channeling waste cells and materials trapped by the at least one filter away from said filtered biological fluid.

2. The device of claim 1, wherein the biological fluid is blood.

3. The device of claim 1, further comprising at least one pump for moving the biological fluid through said at least one filter.

4. The device of claim 1, wherein the mesh size of one of said at least one filter is 10 to 30 microns.

5. The device of claim 1, comprising at least two filters.

6. The device of claim 1, wherein said input valve connects first to a deoxygenation chamber, said deoxygenation chamber connects to a magnetic field chamber, and said magnetic field chamber has a first output valve connected to said at least one filter to deliver biological fluids to said filter, and a second output valve for channeling non-paramagnetic cells and materials away from said biological fluid, such that said biological fluid is subjected to deoxygenation followed by magnetic separation of non-paramagnetic cells and materials from said biological fluid prior to delivery of said biological fluid into said at least one filter.

7. The device of claim 6, further comprising a system integrated between the deoxygenation chamber and the magnetic field chamber, which system cools the deoxygenated biological fluid prior to passage into said magnetic field chamber.

8. The device of claim 6, further comprising an oxygenation chamber connected to the first output valve of the magnetic field chamber to re-oxygenate said biological fluid following magnetic separation.

9. The device of claim 5, comprising at least two parallel filtration chambers with time-alternating filtration and waste destruction functions such that at least one filter is operational at all times.

10. The device of claim 1, further comprising homogenization of waste cells and materials trapped by the at least one filter, wherein said homogenization is achieved by administration of one or more of ultrasonic pulses, heat, cold, radiation, electrocution, and mechanical milling or grinding to said waste cells and materials trapped by said filter.

11. The device of claim 9, wherein the output valves of said at least two parallel filtration chambers connect to a mitotic cell damaging chamber, such that said filtered biological fluid is subjected to mitotic cell damaging treatment prior to return of said biological fluid to said subject.

12. The device of claim 1, wherein said input valve connects first to a mitotic cell damaging chamber having an output valve connected to said at least one filter, such that said biological fluid is subjected to mitotic cell damaging treatment prior to delivery of said biological fluid into said at least one filter.

13. The device of claim 11, wherein the mitotic cell damaging treatment is selected from one or more of radiation, UV light, radiowave frequencies, ultrasonic pulses, heat, cold, or hypoxia.

14. A device for filtering fluid-borne cancer cells from the biological fluid of a subject with cancer, the device comprising: (a) at least one affinity column that binds one or more cancer antigens; (b) an input valve for delivery of biological fluid from said subject into said at least one affinity column; (c) an affinity column output valve from said at least one affinity column to deliver the filtered biological fluid back into said subject.

15. The device of claim 14, comprising at least two affinity columns operating in parallel.

16. The device of claim 15, wherein the output valves of said at least two parallel affinity columns connect to at least two parallel filtration chambers with time-alternating filtration and waste destruction functions such that at least one filter is operational at all times.

17. The device of claim 1, integrated in microscale on a silicon or other semiconducting substrate.

18. A method for filtering fluid-borne cancer cells from the biological fluid of a subject with cancer, the method comprising filtering the biological fluid of said subject through at least one filtration device and returning filtered biological fluid back to said subject, wherein the device comprises: (a) an input valve for delivery of said biological fluid from said subject into at least one filter; (b) a first filter output valve from said at least one filter to return the filtered biological fluid back into said subject; and (c) a second filter output valve from said at least one filter for channeling waste cells and materials trapped by the at least one filter away from said filtered biological fluid.

19. The method of claim 18, wherein the biological fluid is blood.

20. The method of claim 18, wherein the device further comprises at least one pump for moving the biological fluid through said at least one filter.

21. The method of claim 18, wherein the mesh size of one of said at least one filter is 10 to 30 microns.

22. The method of claim 18, wherein the device comprises at least two filters.

23. The method of claim 18, wherein the device comprises at least two parallel filtration chambers with time-alternating filtration and waste destruction functions such that at least one filter is operational at all times.

24. The method of claim 18, wherein said input valve of said device connects first to a deoxygenation chamber, said deoxygenation chamber connects to a magnetic field chamber, and said magnetic field chamber has a first output valve connected to said at least one filter to deliver biological fluids to said filter, and a second output valve for channeling non-paramagnetic cells and materials away from said biological fluid, such that said biological fluid is subjected to deoxygenation followed by magnetic separation of non-paramagnetic cells and materials from said biological fluid prior to delivery of said biological fluid into said at least one filter.

25. The method of claim 24, wherein the device further comprises a system integrated between the deoxygenation chamber and the magnetic field chamber, which system cools the deoxygenated biological fluid prior to passage into said magnetic field chamber.

26. The method of claim 24, wherein the device further comprises an oxygenation chamber connected to the first output valve of the magnetic field chamber to re-oxygenate said biological fluid following magnetic separation.

27. The method of claim 18, wherein the device comprises homogenization of waste cells and materials trapped by the at least one filter, wherein said homogenization is achieved by administration of one or more of ultrasonic pulses, heat, cold, radiation, electrocution, and mechanical milling or grinding to said waste cells and materials trapped by said filter.

28. The method of claim 23, wherein the output valves of said at least two parallel filtration chambers connect to a mitotic cell damaging chamber, such that said filtered biological fluid is subjected to mitotic cell damaging treatment prior to return of said biological fluid to said subject.

29. The method of claim 18, wherein said input valve of said device connects first to a mitotic cell damaging chamber having an output valve connected to said at least one filter, such that said biological fluid is subjected to mitotic cell damaging treatment prior to delivery of said biological fluid into said at least one filter.

30. The method of claim 29, wherein the mitotic cell damaging treatment is selected from one or more of radiation, UV light, radiowave frequencies, ultrasonic pulses, heat, cold, or hypoxia.

31. A method for filtering fluid-borne cancer cells from the biological fluid of a subject with cancer comprising filtering said biological fluid through at least one filtration device and returning filtered biological fluid back to said subject, wherein said device comprises: (a) at least one affinity column that binds one or more cancer antigens; (b) an input valve for delivery of biological fluid from said subject into said at least one affinity column; and (c) an affinity column output valve from said at least one affinity column to deliver the filtered biological fluid back into said subject.

32. The method of claim 31, wherein the device comprises at least two affinity columns operating in parallel.

33. The method of claim 32, wherein the output valves of said at least two parallel affinity columns connect to at least two parallel filtration chambers with time-alternating filtration and waste destruction functions such that at least one filter is operational at all times.

34. The method of claim 18, wherein said device is integrated in microscale on a silicon or other semiconducting substrate.
